(19) United States
(12) Patent Application Publication
(10) Pub. No.: US 2024/0228580 A1
(43) Pub. Date: Jul. 11, 2024

(54) DENDRITIC CELL-DERIVED VESICLES AND ACTIVATION OF ANTIGEN-SPECIFIC T-CELLS

(71) Applicant: University of Kentucky Research Foundation, Lexington, KY (US)

(72) Inventors: Jill M. Kolesar, Lexington, KY (US); Christopher I. Richards, Lexington, KY (US)

(21) Appl. No.: 18/405,778

(22) Filed: Jan. 5, 2024

Related U.S. Application Data
(60) Provisional application No. 63/478,635, filed on Jan. 5, 2023.

(51) Int. Cl. C07K 14/74; A61K 9/127; A61K 35/15; C12N 5/0783; C12N 5/0784

(52) U.S. Cl. CPC C07K 14/70539; A61K 9/127; A61K 35/15; C12N 5/0636; C12N 5/0639

(57) ABSTRACT

An antigen-presenting dendritic cell membrane derived nanovesicle (CDNV) can be made by incubating a dendritic cell (DC) with an antigen and an agent to activate the DC, thereby generating a mature DC displaying a major histocompatibility complex class I (MHC) presenting the antigen, and fragmenting the membrane of the mature DC and allowing the fragmented membrane to assemble into a CDNV displaying the MHC presenting the antigen. The CDNV can be delivered to an environment including a T cell, thereby directly activating the T cell, or indirectly activating the T cell through a bystander antigen presenting cell (APC) that uptakes the CDNV and presents the antigen. The activated T cell and produce a T cell response.

### RELATED APPLICATIONS

This application claims priority from U.S. Provisional Application Ser. No. 63/478,635 filed Jan. 5, 2023, the entire disclosure of which is incorporated herein by this reference.

### TECHNICAL FIELD

The presently-disclosed subject matter generally relates to compositions and methods for use in activating antigen-specific T-cells. Certain embodiments of the presently-disclosed subject matter relate to preparation of cell-derived vesicles and activation of antigen-specific T-cells. Certain embodiments of the presently-disclosed subject matter relate to use of dendritic cell membrane-derived nanovesicles for T-cell activation.

### INTRODUCTION

T cells play an integral role in the generation of a sufficient and effective immune response. As a component of the adaptive immune system, T cells work to clear foreign microbes that have bypassed defenses of the innate immune system. (1) T cells are activated when they encounter foreign antigens presented on the surface of antigen-presenting cells (APCs). This recognition occurs through the interaction of the T-cell receptor (TCR) on the T-cell with an antigen-Major Histocompatibility Complex (MHC) on the APC.

Upon activation, CD8⁺ T cells gain cytotoxic capabilities, enabling elimination of not only cells infected with intracellular microbes, but tumor cells as well. (2) In particular, the CD8⁺ T cells recognize and bind to target cells presenting the foreign antigen-MHC molecules. Upon binding, they release cytotoxic substances, which induce apoptosis in the target cell.

The ability of CD8⁺ T cells to target and eliminate tumor cells has led to the development of potential therapeutic strategies that harness this capability. While cell-based immunomodulation has shown to be effective to some degree in patients with B cell lymphoma and malignant melanoma, clinical application is limited due to lack of long-term storage stability, risk of in vivo replication and lodging in microvasculature, and susceptibility to immunosuppression. (3-5)

An alternative approach is the utilization of extracellular vesicles (EVs) secreted from immune cells that are capable of performing antigen presentation for T cell activation, as they still maintain MHCs. For example, exosomes, a subgroup of EVs, possess lipid bilayer membranes that mimic the composition of the parental cell, retaining functional membrane proteins that enable homing to, and interaction with, target cells. (6-12) Exosomes have been found to possess a variety of functional characteristics that have been utilized for therapeutic applications. Such applications include cargo transport between cells, induction of angiogenesis, and immune modulation, rendering them ideal for immunotherapy. (6,13-16)

Exosomes secreted by APCs, namely, dendritic cells (DC), possess biologically functional MHC surface molecules presenting antigenic peptides (pMHC) capable of inducing antigen-specific T cell responses. (4,13,17-21) When DCs are treated with tumor-associated antigens (TAAs), secreted exosomes retain the ability of parental DC to present these antigens and activate TAA-specific T cells, inducing a TAA-targeted response. (4,5,20,22,23) The function or immunomodulatory ability of exosomes can be further altered or enhanced through surface modification (e.g., amino conjugation, lipid insertion, and PEG (polyethylene glycol)-ylation) or genetic engineering of the parental cell or exosome itself to increase target specificity or load exogenous or endogenous molecules onto the exosome surface or interior. (24,25)

Although exosome-based cancer immunotherapy has progressed, clinical application is hindered due to manufacturing constraints, such as low yields and a lack of scalable production, giving rise to investigation into alternative, exosome-mimicking platforms. (16,26-30) In an attempt to overcome these obstacles, synthetic alternatives to exosomes have been explored and include systems such as liposomes, dendrimers, nanogels, and metallic nanoparticles. Such systems may possess attributes that enable scalable production, high yields, customizable composition, or modifiable physiochemical properties but are hindered by cost and aggregation during storage and, due to their exogenous nature, lack intrinsic targeting capabilities and can be immunogenic or toxic. (13,16,25,29,31-34)

Biomimetic hybrid platforms that employ a synthetic nanoparticle core coated with a cell membrane have been proposed and possess desirable traits, as well as having shown favorable results in mice. (35,36) However, much like exosomes, such platforms are impeded by difficulty in achieving large-scale production, in addition to poor reproducibility and low efficiency in coating the nanoparticle core with cell membrane. (35,37)

Cell-derived nanovesicles (CDNVs), which are artificially generated through fragmentation of cell membranes, have shown similar properties as exosomes, including target-specific cargo delivery and the incorporation of peptide-presenting MHC molecules on the CDNV surface to facilitate T cell activation via direct or indirect mechanisms. (38-42) Studies using dendritic cell-derived nanovesicles primarily focused on mediating T cell activation by inducing fusion or aggregation of CDNVs or by simultaneous treatment with CDNVs and free peptides. (38,39) The most commonly used methods of CDNV production rely on ultrasonic or friction/shearing techniques, such as extrusion, that are low throughput, have limited scalability, or denature proteins through generation of high levels of heat. (43,44)

Accordingly, there remains a need in the art for improved compositions and methods for use in immunotherapy and for mediating T cell activation.

### SUMMARY

The presently-disclosed subject matter meets some or all of the above-identified needs, as will become evident to those of ordinary skill in the art after a study of information provided in this document.

T cells play an integral role in the generation of an effective immune response and are responsible for clearing foreign microbes that have bypassed innate immune system defenses and possess cognate antigens. The immune response can be directed toward a desired target through the selective priming and activation of T cells. T cells are activated when they encounter foreign antigens presented on the surface of antigen-presenting cells (APCs). This recognition occurs through the interaction of the T-cell receptor (TCR) on the T-cell with an antigen-Major Histocompatibility Complex (MHC) on the APC.

Dendritic cells and macrophages are both examples of APCs. They have a number of common functions, such as phagocytosis and antigen presentation; however, they also have distinct roles and characteristic.

Macrophages originate from monocytes and are found throughout the body in almost all tissues, where they patrol and maintain tissue integrity. Macrophages adapt to the local environment, taking on specialized functions depending on their tissue location. Meanwhile, dendritic cells are primarily derived from bone marrow precursors and are commonly found in peripheral tissues, especially those that have contact with the external environment, like the skin and the lining of the nose, lungs, stomach, and intestines. They are also present in the lymph nodes and bloodstream.

With regard to immune response, macrophages are primarily involved in the early stages. They are adept at engulfing and digesting pathogens and cellular debris. While they do present antigens to T cells, their primary function is more aligned with clearing infections and initiating inflammation. Dendritic Cells are considered the most potent of the APCs. Their primary role is to process antigen material and present it on their surface to T cells in the lymph nodes. This makes them key players in activating the adaptive immune response.

Regarding antigen presentation, macrophages present antigens to T cells as part of their role in the innate immune system, but they are generally less efficient in activating naïve T cells compared to dendritic cells. Dendritic cells excel in capturing antigens in peripheral tissues, migrating to the lymph nodes, and presenting these antigens to naïve T cells. They are particularly useful for the initiation of a primary immune response.

In connection with function and activation, macrophages are involved in both causing and resolving inflammation. They can switch from an inflammatory state (M1, where they kill pathogens and stimulate inflammation) to an anti-inflammatory state (M2, where they promote tissue repair). Dendritic cells, once they have captured antigens, undergo a process of maturation. In their mature state, they have an enhanced ability to activate T cells and are less focused on phagocytosis.

The presently-disclosed subject matter harnesses the ability of dendritic cell (DC) to activate T cell response. Due to this ability, DC membrane derived nanovesicle (CDNV) as disclosed herein have utility in the context of activating antigen-specific T cells, which can occur through both direct and indirect mechanisms. As will be recognized by one of ordinary skill in the art, the ability to affect T cell activation is useful for immunotherapy applications, targeting tumor cells or pathogen-infected cells.

The presently disclosed subject matter includes methods of making an antigen-presenting CDNV, methods of activating an antigen-specific T cell, CDNVs and compositions containing such CDNVs, and use of such CDNVs and compositions for immunotherapy and other applications to target cells of interest presenting the antigen.

Reference is made to FIG. 1 and FIG. 2, which provide schematic representations of the methods and CDNVs as disclosed herein. Briefly, a DC (immature) is incubated with an antigen and an agent that activates the DC, thereby generating a mature DC displaying a major histocompatibility complex class I (MHC) presenting the antigen. The membrane of the mature DC is fragmented and the fragmented membrane is allowed to assemble into a CDNV displaying the MHC presenting the antigen. Indeed, the CDNV possesses a membrane with a similar composition to that of the parental mature DC and are thus capable of performing many of the same biological functions, such as T cell stimulation. Once it has been placed in an environment with a T cell, the CDNV can directly activate the T cell (pathway depicted at the top of FIG. 1 and FIG. 2), or indirectly activating the T cell (pathway depicted at the bottom of FIG. 1 and FIG. 2), through a bystander antigen presenting cell that uptakes the CDNV and presents the antigen. Once the T cell has been activated, it can proceed with the T cell response.

With further reference to the exemplary schematic provided in FIG. 2, in direct T cell activation (FIG. 2, top), CDNVs interact directly with neighboring CD8+ T cells and provide activation signals via peptide-MHC (pMHC) class I complexes and costimulatory molecules (CD80/CD86) residing on the CDNV membrane surface with T cell TCR and CD28, respectively. Indirect activation (FIG. 2, bottom) is mediated by bystander APCs that take up CDNVs and subsequently process and cross-present antigenic peptide in the cells' own MHC class I complex. Alternatively, APCs may acquire preformed pMHC class I molecules from CDNVs through the transfer of membrane from CDNV to APC in a process termed as cross-dressing. Through these processes, the ability to activate neighboring CD8⁺ T cells may be transferred from CDNV to recipient APC.

As disclosed herein, the presently-disclosed subject matter harnesses the ability of DCs to process and present antigens, and further utilizes fragmentation of the cell membrane to generate cell membrane-derived nanovesicles that retain the composition of the mature DC precursor cell membrane. The resulting CDNVs possess the capability to present antigens and facilitate activation of antigen-specific T cells by both direct interaction with T cells and indirect activation through uptake by bystander APCs that then activate T cells. Not only do the CDNVs disclosed herein retain the necessary surface molecules required for T cell activation, but also CDNVs generated as described herein can be produced in higher yields while being less labor-intensive than exosomes. Furthermore, the production efficiency of CDNVs is enhanced following maturation, increasing the yield of CDNVs with higher T cell activation efficacy in contrast to exosomes, which suffer from a decrease in yield following maturation. Thus, DC cell membrane-derived nanovesicles generated by nitrogen cavitation offer a platform for immunotherapy to surmount limitations associated with cell-, exosome-, and synthetic nanoparticle-based methods.

This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Schematic illustrating the ability of dendritic cells to activate T cell response.

FIG. 2. Schematic illustrating mechanisms of CD8+ T cell activation mediated by DC membrane-derived nanovesicles.

FIG. 3. Phenotypic characterization of unstimulated and IFN-γ-stimulated DC2.4 cells. DC2.4 cells were untreated or treated with 20 ng/mL IFN-γ overnight. Expression of CD11c, CD40, CD54, CD80, CD86, MHC class I, MHC class II, and MHC class I-bound SIINFEKL peptide (red) by untreated and IFN-γ treated DC2.4 was analyzed by flow cytometry. Isotype-matched antibodies (black) were used as controls. Following IFN-γ stimulation, DC2.4 expression of the lineage marker CD11c remained constant, and that of maturation markers CD40, CD54, CD80, CD86, and MHC class I increased, while that of the maturation marker MHC class II decreased. Presentation of SIINFEKL peptide by MHC class I also increased following IFN-γ stimulation.

FIG. 4. Characterization of CDNV surface proteins by western blot. Western blot of precursor DC2.4 cell and DC2.4-derived CDNVs incubated with 5 g/mL SIINFEKL peptide and with or without 20 ng/mL IFN-γ overnight. Molecules required for T cell activation, namely, CD80, CD86, and SIINFEKL-presenting MHC class I, were observed on CDNVs from both immature and mature parental DC2.4 cells.

FIG. 5A-5B. NTA characterization of isolated DC2.4 cell CDNVs following incubation of DC2.4 cells with or without IFN-γ. DC2.4 cells were left untreated or pulsed with 20 ng/mL IFN-γ overnight. Cell membrane-derived nanovesicles were generated by fragmentation of the cell membrane via nitrogen cavitation at 300 psi for 15 min and isolated by differential centrifugation at 500 g, 2000 g, 10,000 g, and 100,000 g. The CDNV size was measured by nanoparticle tracking analysis (NTA) and NTA3.4 software. (A) Size distribution of CDNVs isolated per 1×10⁶ unstimulated (solid line) or IFN-γ-stimulated (dashed line) precursor DC2.4 cells, with average diameters of 123±1 and 125±2 nm, respectively. (B) Total yield of CDNVs per 1×10⁶ unstimulated (1.31×10¹⁰±0.9×10⁹) (red) or IFN-γ-stimulated (2.0×10¹⁰±2×10⁹) (blue) precursor DC2.4 cells (mean±SEM, n=6). *p<0.05.

FIG. 6A-6C. Field-emission scanning electron microscopy images of CDNVs generated from DC2.4 cells. CDNVs generated via nitrogen cavitation and analyzed by feSEM were measured to have a diameter of ˜60-300 nm. CDNV morphology was observed to be highly spherical (FIG. 6A, 6B) or spherical with some degree of irregularity (FIG. 6C). The irregular spherical shape of CDNVs may be a result of sample preparation for feSEM.

FIG. 7A-7C. SIINFEKL-presenting CDNVs promote direct activation of T cells. FIG. 7A: Schematic demonstrating the proposed mechanism of CDNV production and direct activation of T cells. In this approach, immature DC2.4 cells are pulsed with 20 ng/mL IFN-γ to stimulate maturation and 5 μg/mL SIINFEKL peptide for cross-presentation. CDNVs are then generated from mature, peptide-presenting DC2.4 cells by nitrogen cavitation and isolated via differential ultracentrifugation. Direct CD8⁺ T cell activation is mediated by interaction of pMHC complexes and costimulatory molecules on CDNV_{SIIN}⁺/IFN-γ⁺ with TCR and costimulatory receptors on recipient CD8⁺ T cells. FIG. 7B: Phenotypic analysis of the T cell early activation marker CD69 following CD8⁺ T cell treatment with CDNVs generated from immature (IFN-γ) or mature (IFN-γ+) DCs incubated with (SIIN) or without (SIIN) SIINFEKL peptide. FIG. 7C: Expression of CD69, measured as the geometric mean fluorescence intensity (Geo. MFI) following incubation of CDNVs generated from immature (IFN-γ) or mature (IFN-γ) DCs incubated with (SIIN) or without (SIIN) SIINFEKL peptide. Statistical analysis was performed using one-way ANOVA with Tukey's HSD test comparing immature to mature CDNVs (*p>0.05,**p>0.01, and***p>0.001) or CDNV mixture (#p>0.05, ##p>0.01, and ###p>0.001) (mean±SEM, n=3).

FIG. 8A-8C. SIINFEKL-presenting CDNVs promote indirect activation of T cells through delivery of antigen to recipient APCs. FIG. 8A: Schematic demonstrating the mechanism of CDNV production and delivery of antigenic peptide to bystander APCs, conferring the ability to activate T cells. As before, immature DC2.4 cells were pulsed with 20 ng/mL IFN-γ and 5 μg/mL SIINFEKL peptide, which then underwent nitrogen cavitation and differential centrifugation to generate and isolate peptide-presenting CDNVs. Upon DC2.4 treatment with peptide-presenting CDNVs, recipient DC2.4 cells may take up and present antigenic peptides in MHC I complexes on their surface through cross-presentation or cross-dressing. Antigenic peptide-presenting DC2.4 cells then interact with naïve CD8⁺ T cells, stimulating T cell activation. FIG. 8B: Phenotypic analysis of T cell activation by T cell early activation marker CD69 expression following CD8⁺ T cell treatment with DC2.4 cells pulsed with CDNV_{SIIN}⁺/IFN-γ⁻, CDNV_{SIIN}⁺/IFN-γ⁺, or 1:1 mixture of CDNV_{SIIN}⁺/IFN-γ⁺ CDNV_{SIIN}⁺/IFN-γ (CDNV_{Mix}). FIG. 8C: Expression of the early T cell activation marker CD69, measured as Geo. MFI, following incubation with DC2.4 cells pulsed with CDNV_{SIIN}⁺/IFN-γ, CDNV_{SIIN}⁺/IFN-γ⁺, or CDNV_{Mix}. Statistical analysis was performed using one-way ANOVA with Tukey's HSD test comparing immature to mature CDNVs (*p>0.05, **p>0.01, and***p>0.001) or CDNV mixture (#p>0.05, ##p>0.01, and ###p>0.001) (mean±SEM, n=3).

FIG. 9A-9B. DC2.4 CDNVs are taken up by bystander DC2.4 cells primarily through clathrin-mediated routes. FIG. 9A: DC2.4 cell membranes were labeled with lipophilic membrane stain DiO. CDNVs from DC2.4 cells were labeled with DiI. Bystander DiO-labeled DC2.4 cells were pretreated with or without 80 μM clathrin-mediated endocytosis inhibitor Dynasore for 30 min. DiO-labeled DC2.4 cells were then cultured with 5×10¹⁰ DiI-labeled CDNVs with or without Dynasore for 3 h and imaged by confocal microscopy. Cells in 0.4% DMSO were used as the control. FIG. 9B: CDNV uptake by bystander APCs was measured by the fluorescence of cellular DiI. The integrated density (ID) was calculated from fluorescence images. Statistical analysis was performed using one-way ANOVA with Tukey's HSD test (***p>0.001) (mean±SEM, n=9).

FIG. 10. CDNV effect on IL-6 cytokine production of recipient DC2.4 cells. DC2.4 cells were cultured with CDNVs generated from immature DC2.4 (CDNV_{IFN}-γ⁻) or mature DC2.4 (CDNV_{IFN}-γ⁺) cells for 18 h, and IL-6 production was measured via ELISA. Statistical analysis was performed using one-way ANOVA with Tukey's HSD test (mean±SEM, n=3).

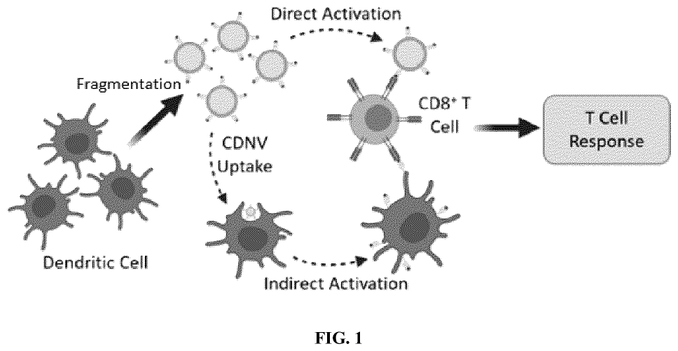

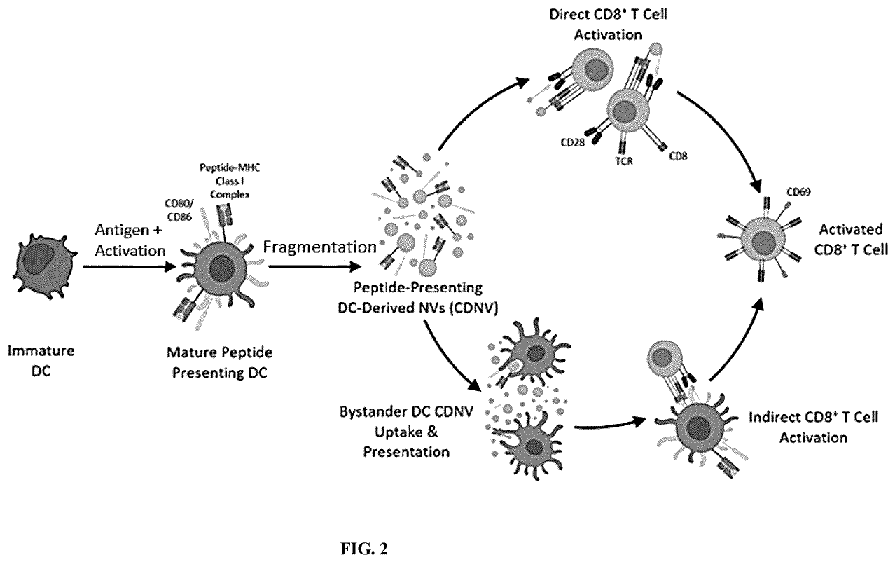

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes a method of making an antigen-presenting dendritic cell membrane derived nanovesicle (CDNV), which includes incubating a dendritic cell (DC) with an antigen and an agent to activate the DC, thereby generating a mature DC displaying a major histocompatibility complex class I (MHC) presenting the antigen, and fragmenting the membrane of the mature DC and allowing the fragmented membrane to assemble into a CDNV displaying the MHC presenting the antigen.

Various agents are known in the art for activating DCs. Examples include, but not limited to, interferon-gamma (IFN-γ), tumor necrosis factor-alpha (TNF-α), interleukin-1 (IL-1), lipopolysaccharides (LPS), CD40 ligand (CD40L), and polyinosinic-polycytidylic acid (Poly I:C). Presence of a pathogen will also activate DCs.

The antigen is an antigen that is presented by a target cell. In this regard, the target cell refers to a cell to which it is desirable to direct a T cell response.

In some embodiments, the target cell is a tumor cell. In some embodiments, the target cell has been obtained from an in vivo environment at a site of a tumor in a subject.

In some embodiments, the target cell is a cell infected with a pathogen. For example, the cell could be infected with bacteria, viruses, fungi, and/or protozoa. In some embodiments, the target cell has been obtained from an in vivo environment at a site of an infection in a subject.

In some embodiments, the target cell is obtained from an in vitro environment or an in vivo environment. For example one might obtain a cell from an in vitro culture. In some cases, the in vitro culture was prepared and propagated from a cell originally obtained from a subject. In some embodiments it is possible that the antigen of interest is a polypeptide or polynucleotide having a known sequence, allowing for use of recombinant technologies to prepare antigen sample for incubation with the DCs.

In some embodiments, the method of making a CDNV can also include obtaining the DC from a circulating hematopeotic cell obtained from a subject. The DC used to make the CDNV can be obtained from a source selected based on the desired application. For example, in some embodiments, the DC is obtained from bone marrow. For another example, in some embodiments, the DC is obtained from circulating hematopeotic cells. Depending on the circumstances, the DC could be obtained from, for example, a subject for whom activation of an antigen-specific T cell is desired, such as a subject in need of treatment for a cancer or a pathogenic infection. For another example, the DC could be obtained from a donor subject, such as a healthy, normal donor subject. In this regard, in some embodiments, circulating hematopeotic cells could be obtained from a subject (patient or donor) by apheresis. As will be appreciated by one of ordinary skill in the art, in this procedure, blood is drawn from the subject and passed through a machine that separates out the hematopoietic stem cells. The remaining blood components can then be returned to the subject's bloodstream.

To fragment the membrane of a cell, various methods are known in the art, such as sonication, cavitation, detergent lysis, osmotic shock, freeze-thaw cycles, and mechanical disruption. As will be appreciated by one of ordinary skill in the art, certain methods should be avoided because they could thwart the ability to generate functional CDNVs, such methods that would denature proteins through generation of high levels of heat.

In some embodiments of the method of making a CDNV, the fragmenting comprises exposing the mature DC to sonication. For example, a cell suspension can be exposed to sonication using an ultrasonic probe or bath. The duration and power settings can impact the size of the resulting vesicles.

In some embodiments of the method of making a CDNV, the fragmenting comprises pressurizing the mature DC under nitrogen and releasing pressure. This process is referred to as nitrogen cavitation. In some embodiments, for example, the pressure under which the mature DC are placed is about 200 to about 500 psi. In this regard, pressures of about 200, 225, 250, 275, 300, 325, 350, or ranges in between could be used. It has been found that higher pressures lead to larger vesicles being assembled, while lower pressures lead to smaller vesicles being assembled. Accordingly, for example, use of a pressure of 200 psi would result in relatively smaller vesicles, while use of a pressure of 500 psi would result in relatively larger vesicles.

In some embodiments, the method of making a CDNV can also include suspending the membrane fragments of the mature DC in an assembly solution. The assembly solution can optionally contain cargo, such that the CDNV encapsulates the cargo during assembly. In such embodiments, the cargo can be selected based on the desired application. For example, cargo could include genetic material, CRISPR, therapeutic agents, protein, and fluorescent markers. In some embodiments, the CDNV has a therapeutic application, and thus, it can be desirable to select a therapeutic agent. For example, if the therapeutic application is to cancer, a chemotherapeutic agent, a checkpoint inhibitor, or an anti-cancer agent could be selected. For another example, in the case of an pathogenic infection, it could be useful to select an antibiotic, antiviral, or other anti-pathogenic agent.

The presently-disclosed subject matter includes a method of activating an antigen-specific T cell, which makes use of a CDNV as disclosed herein. The CDNV is delivered to an environment including a T cell, thereby directly activating the T cell, or indirectly activating the T cell through a bystander antigen presenting cell (APC) that uptakes the CDNV and presents the antigen.

In some embodiments, the CDNV is delivered to an environment by administering the CDNV to a subject. In some embodiments, an effective amount of the CDNV is delivered to the subject for use in treating a cancer. In some embodiments, an effective amount of the CDNV is delivered to the subject for use in treating a pathogenic infection.

There are various methods known in the art for administering a nanovesicle, exosome, or other cell-membrane-based vesicle to a subject. Examples include, but are not limited to, intravenous injection, by which administration is made directly into the bloodstream, commonly used for systemic distribution; intraperitoneal injection, by which administration is made into the peritoneal cavity; inhalation, by which administration targeted delivery can be made to the respiratory system; oral administration, by which gut-targeted can occur; and local injection, by which administration delivery into specific tissues or tumors can be achieved.

In some embodiments of the methods disclosed herein, the CDNVs can be administered to the subject by injection. Various injection methods are known and can be used in accordance with the presently-disclosed subject matter. For example, intravenous injection can be used. For another example, intraperitoneal injection can be used. As will be appreciated by one of ordinary skill in the art, the administration method can be selected by one skilled in the medical arts in view of various circumstances, including the particular subject and/or the particular tumor or infection of interest. For example, in some embodiments in which the subject has ovarian cancer, it can be useful to select intraperitoneal injection.

The presently-disclosed subject matter further includes CDNVs as disclosed herein and compositions comprising such CDNVs. Such compositions can additionally include a pharmaceutically-acceptable carrier and/or other components useful for providing stability, bioavailability, and safety. Examples of such components include, but are not limited to, buffers to maintain pH and osmolarity, stabilizers to protect vesicles during freeze-drying or storage, cryoprotectants to prevent damage during freezing, surfactants to enhance solubility and stability, and preservatives for maintaining sterility and longevity of the formulation.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, compositions, and materials similar or equivalent to those described herein can be used in the practice or testing of the presently-disclosed subject matter, representative methods, compositions, and materials are described herein.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intraperitoneal administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific therapeutic employed and like factors well known in the medical arts. For example, it is well within the skill of the art to start doses at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the terms “polynucleotide” and “nucleic acid molecule” refer to an oligomer or polymer containing at least two linked nucleotides or nucleotide derivatives joined together, typically by phosphodiester linkages. Polynucleotides are inclusive of deoxyribonucleic acid molecules (DNA) and a ribonucleic acid molecules (RNA).

As used herein, the term “polypeptide” means any polymer comprising any of the protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted.

As used herein, the term “subject” refers to a target of administration. The subject of the herein disclosed methods can be a mammal. Thus, the subject of the herein disclosed methods can be a human, non-human primate, horse, pig, rabbit, dog, sheep, goat, cow, cat, or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the terms “treatment” or “treating” relate to any treatment of a condition of interest, including but not limited to prophylactic treatment to prevent development or reduce severity of the condition. The terms “treatment” or “treating” include preventing a condition from occurring in a subject who may be predisposed to the condition but who has not yet been diagnosed as having it; inhibiting the condition, i.e., arresting their development; or ameliorating or relieving the symptoms of the condition, i.e., causing regression of one or more of the symptoms.

The presently-disclosed subject matter is further illustrated by the following specific but non-limiting examples. The following examples may include compilations of data that are representative of data gathered at various times during the course of development and experimentation related to the present invention.

### EXAMPLES

### Cell Culture

The hybridoma CD8⁺ T cell line B3Z was generously provided by Dr. J. Woodward (University of Kentucky Medical Center, KY, USA). B3Z cells were cultured in DMEM (Corning) supplemented with 10% heat-inactivated FBS (Corning), 100 unit/mL penicillin, 100 μg/mL streptomycin, 0.292 mg/mL L-glutamine (Gibco), 0.1 mM non-essential amino acids (Gibco), and 1 mM sodium pyruvate (Gibco). The immature dendritic cell line DC2.4 was purchased from Millipore Sigma. DC2.4 cells were cultured in RPMI 1640 medium (Gibco) supplemented with 10% FBS (Corning), 2 mM L-glutamine (Gibco), 0.1 mM non-essential amino acids (Gibco), 10 mM HEPES buffer (Gibco), and 0.5 mM β-mercaptoethanol (Fisher Scientific) for a maximum of 10 passages. Cells were cultured at 37° C. with 5% CO₂.

### Nanovesicle Production and Isolation

When indicated, DC2.4 cells were cultured with 20 ng/ml recombinant mouse IFN-γ (Invitrogen) with or without 5 μg/mL SIINFEKL peptide (AnaSpec) overnight. Cells were harvested, washed thoroughly with 1×phosphate-buffered saline (PBS), and then resuspended in protease inhibitor buffer solution, comprised of 1 protease inhibitor tablet (Pierce) per 10 mL 1×PBS, at a concentration of approximately 15×10⁶ to 20×10⁶ cells/mL. Cell suspension was then transferred to a prechilled cell disruption vessel (Parr Instrument Company, IL, USA) and pressurized to 300 psi under nitrogen for 15 min to achieve pressure equilibration on ice. A pressure of 300 psi was chosen, as nitrogen cavitation at 300 psi has been shown to generate CDNVs within the size range to exosomes. (45) Pressure was released and cell cavitate was collected and centrifuged at 300 g for 5 min to pellet unfragmented cells. The supernatant was collected, the cell pellet was resuspended in 10 mL of protease inhibitor buffer solution, and nitrogen cavitation was repeated. Cell cavitate was collected, and CDNVs were isolated via differential centrifugation at 500 g for 10 min, 2000 g for 20 min, 10,000 g for 30 min, and 100,000 g for 90 min, all at 4° C. Pelleted CDNVs were resuspended in 250-300 μL of 1×PBS and pipetted thoroughly to break up the pellet. Resuspended CDNVs were centrifuged at 7500 g for 5 min to lightly pellet vesicle aggregates, which were then broken up by additional pipetting. The CDNV suspension was then centrifuged at 7500 g for 10 min to pellet remaining vesicle aggregates and debris. Proteins, protein aggregates, and free SIINFEKL peptide were removed from nanovesicle suspension by a size-exclusion chromatography PD Mini Trap G-25 column (Cytiva) following the manufacturer's directions. The CDNV suspension was then transferred to a microcentrifuge tube, centrifuged at 10,000 g for 10 min to remove any remaining debris or aggregates, and then stored at 4° C. until use.

### Nanoparticle Tracking Analysis

NTA was performed using a NanoSight NS300 (Malvern) instrument. Samples were measured using fixed camera and detection settings across all sessions. Each sample was recorded for 60 s for a total of five repetitions, with greater than 200 tracks per video, at two dilutions. Analysis was performed using NTA3.4 software.

### Western Blot

DC2.4 cells and CDNV pellets were lysed separately in RIPA buffer composed of 150 mM NaCl, 1% Triton X-100, 0.5% sodium deoxycholate, 0.1% SDS, 50 mM Tris (pH 8, adjusted), and 1×protease inhibitor cocktail (Roche). Protein samples were resolved on 12% polyacrylamide gels and then transferred to a nitrocellulose membrane. Equal amounts of protein from various samples were loaded per lane for comparison studies. The nitrocellulose membrane was blocked for 1 h and then incubated with biotinylated anti-mouse CD80 (16-10A1, Biolegend), anti-mouse CD86 (GL-1, Biolegend), or anti-H-2K^{b}-bound SIINFEKL (25-D1.16, Invivogen) primary antibodies under consistent agitation for 1 h at room temperature in a nonreducing environment. The membrane was then washed and incubated with streptavidin-conjugated HRP (Biolegend) secondary antibody under consistent agitation for 1 h. The membrane was then washed, and bands were visualized by chemiluminescence detection (Clarity, Bio-Rad) using a Chemi-Doc (Bio-Rad) instrument.

### Scanning Electron Microscopy

CDNVs were fixed in 2% paraformaldehyde for 45 min and then rinsed with 1×PBS in triplicate. CDNVs were then serially dehydrated in 50, 60, 70, 80, 90, and 100% (200 proof) ethanol for 10 min and resuspended in 200 proof ethanol. Suspended CDNVs were briefly sonicated, and then a droplet of the sample was pipetted and deposited onto silicon wafer. The surface of the sample was then metallized by sputter-coating 5 nm platinum (EM ACE 600, Leica Microsystems, Wetzlar, Germany) to enhance surface electrical conductivity. CDNVs were subsequently imaged using field-emission scanning electron microscopy (feSEM, Helios Nanolab 660, Thermo Fisher Scientific, Hillsboro, OR, USA).

### Antibodies and Flow Cytometry

DC2.4 cells were labeled using the following murine monoclonal antibodies from Biolegend, unless stated otherwise: CD16/32 (93), Brilliant Violet 421-conjugated CD11c (N418), phycoerythrin-conjugated CD8a (53-6.7), allophycocyanin-conjugated H-2K^{b} (AF6-88.5), I-A/I-E (M5/114.15.2), CD40 (1C10, Invitrogen), CD54 (YN1/1.7.4), CD69 (H1.2F3), CD80 (16-10A1), CD86 (GL-1), and H-2K^{b}-bound SIINFEKL (25-D1.16). Cell viability was determined by staining with Live/Dead Fixable Near-IR Stain (Invitrogen). Isotype-matched antibodies were used as controls. Samples were acquired on a FACSymphony A3 (BD Biosciences) instrument and analyzed with FlowJo (BD).

### In Vitro T Cell Activation Assay

To evaluate the ability of CDNVs to activate CD8⁺ T cells, CNDVs were generated from DC2.4 cells incubated with or without 20 ng/ml IFN-γ and/or 5 μg/mL SIINFEKL peptide overnight. To assess direct T cell activation, CDNVs were added to B3Z cells (3×10⁵ cells/well) across a dose range for 18 h with gentle agitation. For indirect T cell activation, CDNVs were added to DC2.4 cells (7.5×10⁴ cells/well) across a dose range for 3 h. DC2.4 cells were thoroughly washed and cocultured with B3Z cells (2×10⁵ cells/well) for 18 h with gentle agitation. T cell activation was determined by the early T cell activation marker CD69 via flow cytometry.

### Bystander APC ELISA Assay

To assess the effect the parental cell maturation status of CDNVs has on bystander APCs, CDNVs were generated from DC2.4 cells incubated with or without 20 ng/mL IFN-γ overnight and added to DC2.4 cells (2×10⁵ cells/well) across a dose range for 18 h with gentle agitation. The culture supernatant was collected, and IL-6 was measured by ELISA (MesoScale) following the manufacturer's instructions.

### Fluorescence Imaging

DC2.4 cells were plated on 35 mm glass-bottom dishes at 1×10⁵ cells/dish and labeled with 2.5 μM DiO (Invitrogen) for 45 min. Resuspended CDNVs were labeled with 5 μM DiI (Invitrogen) for 30 min at 37° C. Cells were treated with or without 80 M Dynasore for 30 min prior to CDNV treatment. Cells were treated with 5×10¹⁰ DiI-labeled CDNVs and incubated for approximately 3 h. Cells were rinsed well and resuspended in L-15 medium (Gibco) for imaging. Cells in 0.4% DMSO (VWR Chemicals) were used as controls.

### Statistical Analysis

Statistical analysis was performed using OriginLab 2021b. Flow cytometry data were expressed as the geometric mean±standard error of the mean (SEM). All other data were expressed as the mean±SEM. Statistical significance was determined by the two-sample 1-test or one-way ANOVA with post hoc Tukey's HSD test when appropriate. Statistical significance was indicated as *p<0.05,**p>0.01, and***p>0.001.

### IFN-γ-Induced DC2.4 Maturation

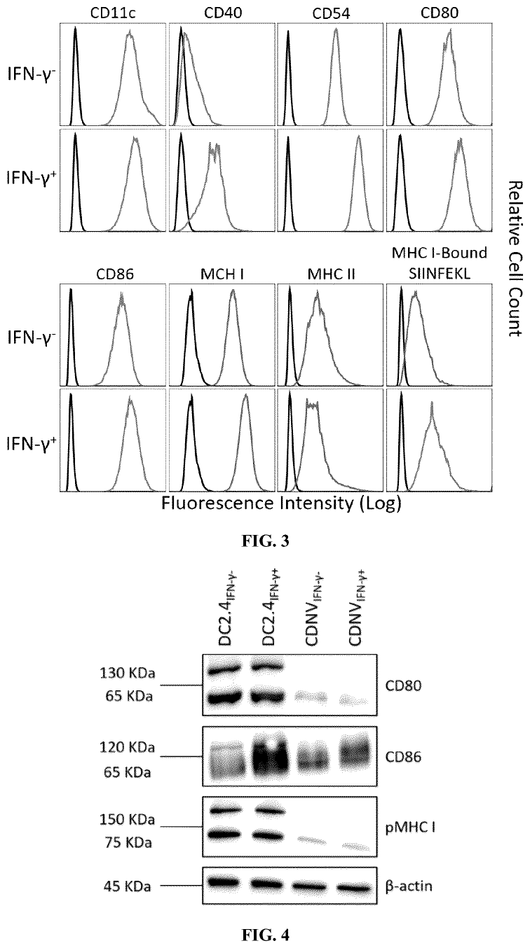

The importance of DC activation to a mature state is demonstrated by the increased ability of mature DCs to activate T cells when compared to their immature counterparts. (46-48) To determine the maturation state of the dendritic cell line DC2.4, the cell phenotype was examined for expression of maturation markers by flow cytometry. As shown in FIG. 3, unstimulated DC2.4 cells were found to have moderate expression of the DC lineage marker CD11c, low expression of the maturation marker CD40, and moderate expression of maturation markers CD54, CD80, CD86, MHC class I, and MHC class II. To activate and induce maturation, DC2.4 cells were cultured with 20 ng/mL IFN-γ overnight. Following incubation with IFN-γ, DC2.4 cells were observed to possess CD11c levels similar to unstimulated cells, while CD40, CD54, CD80, CD86, and MHC class I expression increased, depicted by the increased fluorescence intensity of IFN-γ-treated cells in FIG. 3. However, expression of the maturation marker MHC class II unexpectedly decreased. Although MHC class II decreased, the upregulation of maturation markers CD40, CD45, CD80, and CD86 indicates that unstimulated DC2.4 possesses a relative immature phenotype and acquires a mature phenotype following treatment with IFN-γ.

Activation of CD8⁺ T cells by DCs is MHC class I-restricted, where MHC class I-presented antigenic peptides can be of endogenous or exogenous origin. For example, intracellular infections can result in MHC class I presentation of endogenous peptides, thereby eliciting the targeting of DCs or other cells expressing viral antigens complexed to MHC class I by CD8⁺ T cells. However, DCs can also direct CD8⁺ T cells to target other cells, such as tumors, through internalization, processing, and presentation of exogenous antigens, termed as cross-presentation. (2) To evaluate the ability to cross-present exogenous peptides by immature and mature DC2.4, cells were cultured with 5 μg/mL OVA₂₅₇₋₂₆₄ (SIINFEKL) peptide with or without 20 ng/mL IFN-γ and presentation of MHC class I-bound SIINFEKL was measured by flow cytometry. As shown in FIG. 3, presentation of MHC class I-bound SIINFEKL increased following IFN-γ stimulation. This demonstrates that the treatment of DC2.4 cells with IFN-γ generates mature DC2.4 cells capable of elevated presentation of exogenous peptide by MHC class I. Thus, due to the increased expression of costimulatory molecules CD80/CD86 and the level of MHC class I-bound SIINFEKL by mature DC2.4 cells, CDNVs generated from mature DC2.4 should mediate CD8⁺ T cell activation more efficiently than CDNVs from immature DC2.4 cells.

### CDNV Characterization

As CD8⁺ T cell activation is dependent upon the presence of stimulatory pMHC class I and costimulatory molecules CD80 and CD86, retention of these surface proteins on DC2.4-derived CDNVs was verified by western blot (FIG. 4). The presence of both costimulatory molecules CD80 and CD86, as well as stimulatory MHC class I-bound SIINFEKL, was verified on immature and mature DC2.4, as well as CDNVs generated from immature and mature parental cells. Thus, CDNVs generated from precursor cells by nitrogen cavitation clearly retain the requisite immune-stimulatory molecules for CD8⁺ T cell activation.

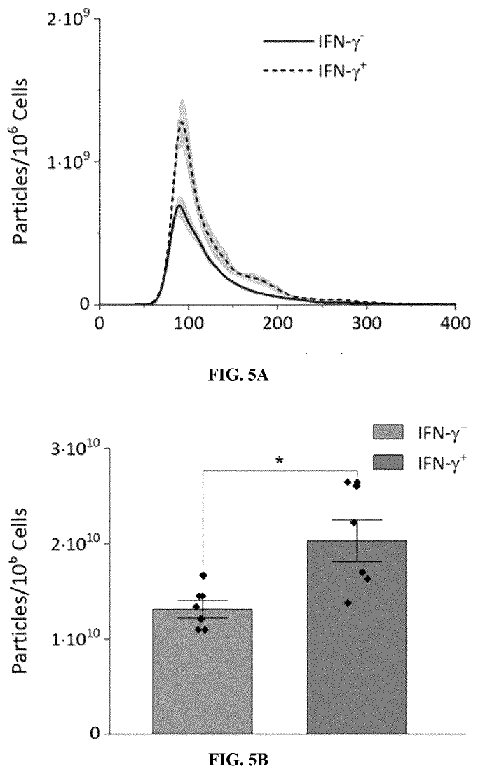

DC maturation is not only associated with changes in protein expression but also morphology. (49,50) To investigate how morphological changes may effect resulting CDNVs or CDNV production, CDNVs from immature and mature DC2.4, generated by nitrogen cavitation and isolated via differential centrifugation, were examined. Hereafter, CDNVs generated from unstimulated and IFN-γ-stimulated DC2.4 cells are referred to as immature (CDNV_{IFN}-γ⁻) and mature (CDNV_{IFN}-γ⁺), respectively. As shown in FIG. 5A, nanoparticle tracking analysis (NTA) found that both CDNV_{IFN}-γ⁻ and CDNV_{IFN}-γ⁺ possess a narrow size distribution, with mean diameters of 123±1 and 125±2 nm, respectively. This observed size range is similar to that of endogenous vesicles, such as exosomes. Next, it was asked if the precursor cell maturation state effects CDNV yield. While it has previously been demonstrated that immature DCs readily produce exosomes, it has also been shown that DCs treated with LPS, to induce maturation, produce 2- to 3-fold fewer exosomes, on average, than immature DCs. (51) Notably, the yield of CDNVs generated from mature DC2.4 cells increases following IFN-γ stimulation. Following DC2.4 maturation, the CDNV yield increased by approximately 50%, from 1.31×10¹⁰±9×10⁸ CDNV_{IFN}-γ per 1×10⁶ unstimulated precursor cells to 2.0×10¹⁰±2×10⁹ CDNV_{IFN}-γ per 1×10⁶ IFN-γ-stimulated precursor cells (FIG. 5B). This increase in CDNV yield following IFN-γ stimulation may be due to morphological changes associated with DC maturation, such as membrane ruffling and formation of dendrites. (49,52) As cell membrane fragmentation by nitrogen cavitation disrupts the cell membrane, which then leads to the formation of small vesicles, these morphological changes may increase the production of CDNVs by increasing the cell membrane surface area available for formation of CDNVs.

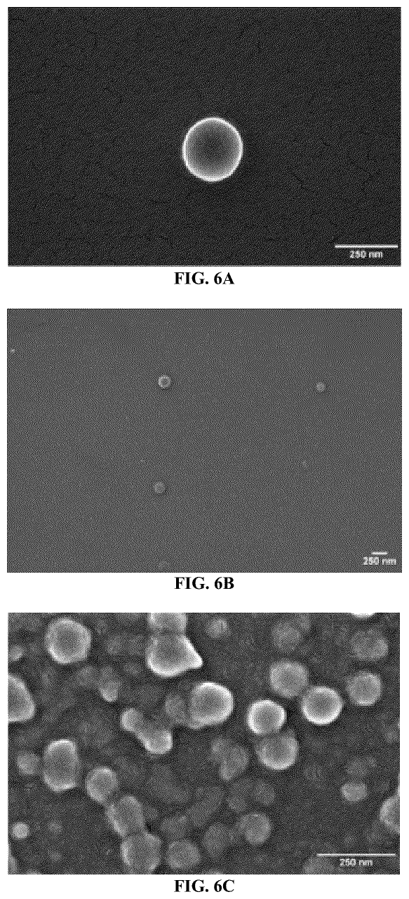

CDNVs were further characterized by field-emission scanning electron microscopy (feSEM), as shown in FIG. 6A-6C. feSEM was found to corroborate the size of nitrogen cavitation-generated CDNVs measured by NTA, as the diameter of CDNV was measured to range from approximately 60 to 300 nm. The morphology of CDNVs was observed to be mostly spherical, with some irregularity. The irregular spherical shape of CDNVs may be a result of sample preparation, as it has been reported that fixation and dehydration may alter the vesicle shape. (53-55)

### Direct CDNV T Cell Activation

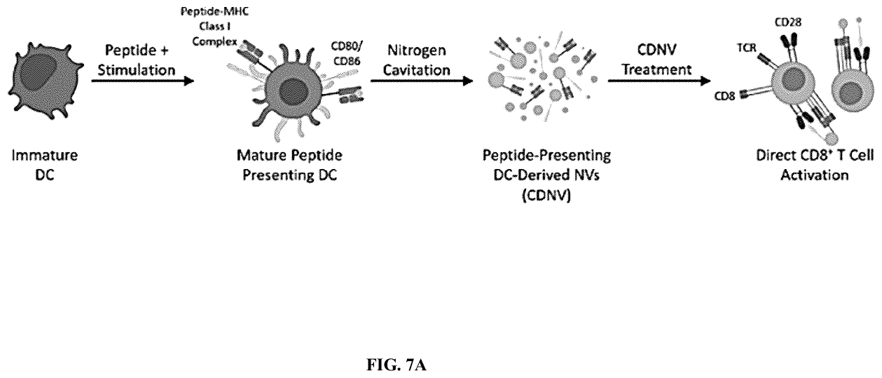

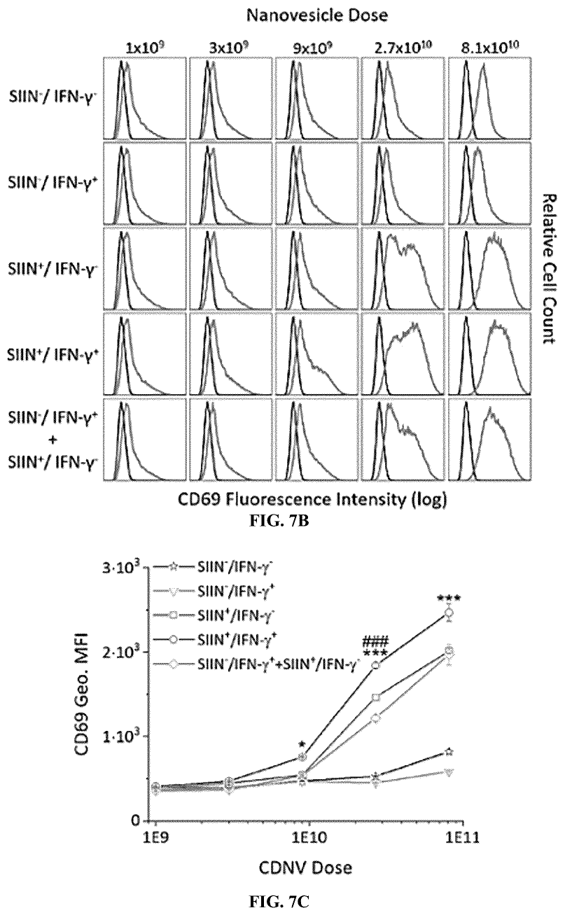

Having confirmed the presence of immune-stimulatory molecules on CDNVs by western blot (FIG. 4), the ability of CDNVs generated from DC2.4 cells to stimulate antigen-specific CD8⁺ T cells via a direct activation mechanism was investigated. As shown in FIG. 7A, direct activation of T cells by CDNVs involves stimulatory pMHC complexes and costimulatory molecules of the CDNV membrane interacting with the T cell receptor (TCR) and T cell costimulatory receptors, providing signaling that induces T cell activation. Here, CDNVs were generated from DC2.4 cells incubated with or without (CDNV_{SIIN}⁺ or CDNV_{SIIN}⁻, respectively) the model peptide SIINFEKL and/or IFN-γ. Isolated CDNVs were incubated with the SIINFEKL-specific, naïve CD8⁺ T cell line B3Z for 16 h. T cell activation was evaluated by expression of the early activation marker CD69. (56) To determine if CDNVs themselves are capable of activating CD8⁺ T cells in the absence of SIINFEKL peptide, CDNVs were generated from untreated DC2.4 cells (CDNV_{SIIN}/IFN-γ). Upon treatment of CD8⁺ T cells with CDNV_{SIIN}/IFN-γ, the population of CD8⁺ T cells with low levels of CD69 expression (CD691) (FIG. 7B, top row) and geometric mean fluorescence intensity (MFI_{Geo}) of T cell CD69 (FIG. 7C), representative of the level of T cell activation, was observed to remain constant at doses of 1×10⁹ (399±5) and 3×10⁹ (393±1) CDNV_{SIIN}/IFN-γ. Relatively small increases in CD69 MFI_{Geo} were observed following CD8⁺ T cell treatment with CDNV_{SIIN}/IFN-γ at doses of 9×10⁹ (475±4), 2.7×10¹⁰ (529±3), and 8.1×10¹⁰ (820±30). While CD69 MFI_{Geo} trended slightly upward with increasing CDNV_{SIIN}/IFN-γ dose, the change in MFI_{Geo} was not statistically significant. To establish that CD8⁺ T cell activation is facilitated by stimulatory and costimulatory signals provided by CDNVs, and not directly mediated by a mature CDNV phenotype, DC2.4 cells incubated with only IFN-γ were used to generate mature, non-SIINFEKL-presenting CDNVs (CDNV_{SIIN}/IFN-γ⁺). Similar to treatment with CDNV_{SIIN}/IFN-γ, the population of CD691.0 (FIG. 7B, second row) and CD69 MFI_{Geo} (FIG. 7C) of CDNV_{SIIN}/IFN-γ⁺-treated CD8⁺ T cells remained relatively constant, with a slight yet nonsignificant trend upward following treatment with 1×10⁹ (361±8), 3×10⁹ (400±10), 9×10⁹ (470±4), 2.7×10¹⁰ (454±3), and 8.1×10¹⁰ (586±6) CDNV_{SIIN}/IFN-γ. Little change in the expression of the early activation marker CD69 was observed by treatment with CDNVs from both non-SIINFEKL-presenting immature and mature DC2.4 cells, suggesting that CDNVs lacking antigenic peptide possess relatively low immunogenicity.

With the observation that CDNVs are relatively non-immunogenic in the absence of antigenic peptide, the capability of antigen-presenting CDNVs to stimulate CD8⁺ T cell activation was investigated by pulsing DC2.4 cells with SIINFEKL peptide with or without IFN-γ. When treated with CDNVs generated from immature, SIINFEKL-presenting DC2.4 (CDNV_{SIIN}⁺/IFN-γ⁻), minimal change in CD691^{Lo} population (FIG. 7B, third row) and CD69 MFI_{Geo} (FIG. 7C) was observed at the 1×10⁹, 3×10⁹, and 9×10⁹ doses of CDNV_{SIIN}⁺/IFN-γ⁻(389±9, 450±16, and 540±25, respectively). A significant increase in the population of T cells with elevated CD69 expression (CD69^{Hi}) was observed upon T cell treatment with 2.7×10¹⁰ CDNV_{SIIN}⁺/IFN-γ⁻(MFI_{Geo} of 1470±37), eliciting T cell populations of CD69^{Lo} and CD69^{Hi}. Expression of CD69 peaked at the maximum dose of 8.1×10¹⁰ CDNV_{SIIN}⁺/IFN-γ⁻(MFI_{Geo} of 2010±32), merging the two T cell populations into a single CD69^{Hi} population, representing activation of the majority of CD8⁺ T cells.

When treated with mature, SIINFEKL-presenting CDNVs (CDNV_{SIIN}/IFN-γ) produced from IFN-γ-stimulated, SIINFEKL-pulsed DCs at doses of 1×10⁹ and 3×10⁹ CDNV_{SIIN}⁺/IFN-γ⁺, CD8⁺ T cell CD69 expression remained low, with CD69 MFI_{Geo} of 412+1 and 470±12, respectively. The level of T cell activation was observed to progressively increase, indicated by the population shift toward CD69^{Hi} (FIG. 7B, fourth row) and an increase in CD69 MFI_{Geo} (FIG. 7C), following treatment with 9×10CDNV_{SIIN}⁺/IFN-γ⁺ (MFI_{Geo} of 757±3), with significant increases in T cell activation and CD69 expression upon treatment with 2.7×10¹⁰ CDNV_{SIIN}⁺/IFN-γ⁺(MFI_{Geo} of 1840±40) and 8.1×10¹⁰ CDNV_{SIIN}⁺/IFN-γ⁻(MFI_{Geo} of 2500±100). Minimal levels of T cell activation occurred after treatment with CDNVs originating from both immature and mature, SIINFEKL-pulsed DC2.4 cells at doses of 1×10⁹ and 3×10⁹ CDNV_{SIIN}⁺/IFN-γ⁻ or CDNV_{SIIN}⁺/IFN-γ⁺. While the level of T cell activation from treatment with 9×10⁹ CDNV_{sIIN}⁺/IFN-γ⁻ remains similar to that observed at lower doses, an increase in T cell activation can be seen at the same dose of 9×10⁹ CDNV_{SIIN}⁺/IFN-γ⁺. Similar to results observed during treatment with CDNV_{SIIN}⁺/IFN-γ, treatment with 2.7×10¹⁰ CDNV_{SIIN}⁺/IFN-γ⁺ yielded a population of both CD691^{Lo} and CD69^{Hi} CD8⁺ T cells; however, while the population of CD691^{Lo} and CD69^{Hi} appear roughly similar when treated with CDNV_{SIIN}⁺/IFN-γ, the T cell population shifted significantly toward CD69^{Hi} following CDNV_{SIIN}⁺/IFN-γ⁺ treatment. At a dose of 8.1×10¹⁰ CDNVs, a single CD69^{Hi} T cell population was observed following treatment with both CDNV_{SIIN}⁺/IFN-γ⁻ and CDNV_{SIIN}⁺/IFN-γ⁺, although CD69 MFI_{Geo} levels were higher in CDNV_{SIIN}⁺/IFN-γ⁺-treated samples. The treatment of T cells with 1×10⁹ and 3×10⁹ CDNV_{SIIN}⁺/IFN-γ was observed to not be statistically more efficient at T cell activation than that with CDNV_{SIIN}/IFN-γ at those same doses. However, when treated with 9×10⁹, 2.7×10¹⁰, or 8.1×10¹⁰ CDNVs, CDNV_{SIIN}/IFN-γ⁺ was a significantly more potent T cell activator than CDNV_{SIIN}⁺/IFN-γ. As expected, these results support that CDNVs generated from mature, SIINFEKL-presenting DC2.4 are more potent activators of CD8⁺ T cells than CDNVs from immature, SIINFEKL-presenting DC2.4 cells in direct CD8⁺ T cell activation. The role of maturation state and subsequent ability of CDNVs to directly activate T cells was further investigated by examining whether stimulatory and costimulatory signals can be provided by separate CDNVs of different maturation status or if activation signals need to originate from the same CDNV. CD8⁺ T cells were incubated with a 1:1 mixture of CDNV_{SIIN}⁺/IFN-γ⁺ CDNV_{SIIN}⁺/IFN-γ⁺ (CDNV_{Mix}). T cells treated with CDNV_{Mix} were observed to have minimal CD69 expression, as determined by CD69 MFI_{Geo}, at doses of 1×10⁹ (357±9) and 3×10⁹ (371±9), with a small increase upon 9× 10⁹ (539±4) CDNV_{Mix} treatment. Following treatment with 2.7×10¹⁰ and 8.1×10¹⁰ CDNV_{Mix}, CD69 expression significantly increased, as CD69 MFI_{Geo} was measured to be 1220±40 and 2000±120, respectively. As shown in FIG. 7B, 7C, T cell activation and CD69 expression mediated by CDNV_{Mix} exhibited a pattern much like that observed in T cells treated with CDNV_{SIIN}⁺/IFN-γ⁻. That is, the relative number of activated T cells and CD69 expression level remained relatively low at doses of 1×10⁹, 3×10⁹, and 9×10⁹, like that observed in CDNV_{sIIN}⁺/IFN-γ⁻-treated samples. Treatment with 2.7×10¹⁰ CDNV_{Mix} resulted in a population composed primarily of CD691R CD8⁺ T cells, with the emergence of CD69^{Hi} CD8⁺ T cells, whereas treatment with 8.1×10¹⁰ CDNV_{Mix} induced a single population comprised primarily of CD69^{Hi} CD8⁺ T cells. Despite the presence of only half the number of SIINFEKL-presenting CDNVs, CDNV_{Mix} was observed to be similarly efficient at T cell activation as CDNV_{SIIN}⁺/IFN-γ⁻, being statistically less efficient at T cell activation only at the dose of 2.7×10¹⁰ CDNVs. These results were interpreted as that although costimulatory signals provided by CDNV_{IFN}-γ⁻ may be reduced relative to CDNV_{IFN}-γ⁺, mature CDNVs may be able to compensate for this signaling deficit by simultaneous interaction with T cells in vitro. However, as facilitation of CD8⁺ T cell activation by CDNV_{SIIN}⁺/IFN-γ⁻ providing stimulatory signaling with CDNV_{SIIN}⁺/IFN-γ⁺ providing costimulatory signaling requires synchronized interaction with the same cell, such a mechanism of T cell activation may not be significant in vivo. Instead, the more likely means of direct T cell activation would occur through stimulatory pMHC and costimulatory signals originating from the same CDNV.

### Indirect CDNV T Cell Activation

CDNVs are capable of presenting peptides and activating T cells directly.

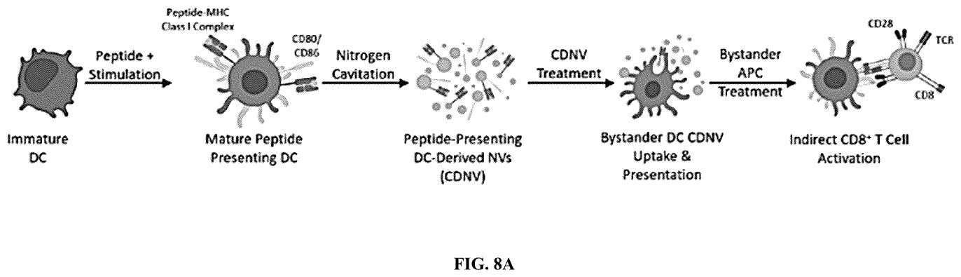

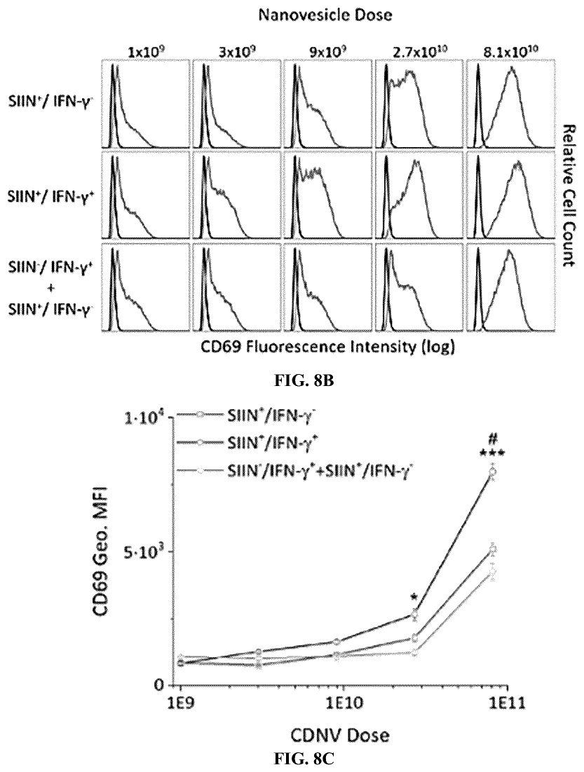

However, CDNV-mediated T cell activation can also occur through indirect means. In contrast to direct activation, indirect activation (FIG. 8A) occurs through the uptake of CDNVs by APCs, which, through cross-presentation or cross-dressing, can present antigenic peptides in MHC class I complexes and facilitate CD8⁺ T cell activation. Previous studies have suggested that in vivo exosome-mediated T cell activation is based on the uptake of vesicles in bystander APCs that then travel to lymph nodes, where they present antigenic peptides and activate cognate T cells. (57) Recent studies have also shown that CDNVs can facilitate the delivery of cargo to target cells and alter their immunological function. (42) To assess the ability of CDNVs to deliver peptide and/or functional pMHC complexes and convey T cell stimulatory ability, DC2.4 cells were pretreated with CDNV_{sIIN}⁺/IFN-γ⁻, CDNV_{SIIN}⁺/IFN-γ⁺, or CDNV_{Mix} for 3 h, washed thoroughly, then cocultured with CD8⁺ T cells. The change in expression of CD69 by the T cell population (FIG. 8B) and CD69 MFI_{Geo} (FIG. 8C) were then examined. As shown in FIG. 8B, 8C, minimal change in T cell activation was observed by treatment of CD8⁺ T cells with DC2.4 cells pretreated with 1×10⁹ (CD69 MFI_{Geo} of 860±40) or 3×10⁹ (CD69 MFI_{Geo} of 768±7) CDNV_{SIIN}⁺/IFN-γ⁻. The population of T cells with elevated CD69 expression marginally increased by treatment with DC2.4 cells pretreated with 9×10⁹ CDNV_{SIIN}⁺/IFN-γ⁻(CD69 MFI_{Geo} of 1160±40), while a significant increase in CD69^{Hi} T cell population by DCs pretreated with 2.7×10¹⁰ (CD69 MFI_{Geo} of 1800±160) and 8.1×10¹⁰ (CD69 MFI_{Geo} of 5100±230) CDNV_{SIIN}⁺/IFN-γ⁻ was observed.

When treated with DCs pretreated with CDNV_{SIIN}⁺/IFN-γ⁺(FIG. 8B, middle row), CD8⁺ T cell activation and CD69 expression were minimal at the lowest dose of 1×10⁹ CDNV_{SIIN}⁺/IFN-γ⁺ (MFI_{Geo} of 768±7). The level of T cell activation mediated by DC2.4 cells was observed to progressively increase with increasing dose of CDNV_{SIIN}⁺/IFN-γ⁺ pretreatment. As the CDNV_{SIIN}⁺/IFN-γ⁺ dose increased, CD69 MFI_{Geo} was observed to be 1280±45 at 3×10⁹, 1640±80 at 9×10⁹, and 2700±230 at 2.7×10¹⁰ CDNV_{SIIN}⁺/IFN-γ⁺, peaking at 8000±300 upon CD8⁺ T cell treatment with DC2.4 cells pretreated with 8.1×10¹⁰ CDNV_{SIIN}⁺/IFN-γ⁺. No statistical difference in T cell activation was observed between CDNV_{SIIN}⁺/IFN-γ and CDNV_{SIIN}⁺/IFN-γ⁺ at doses of 1×10⁹, 3×10⁹, and 9×10⁹, although T cell CD69 MFI_{Geo} tended to be slighter higher from CDNV_{SIIN}⁺/IFN-γ⁺ at doses 3×10⁹ and 9×10⁹ CDNVs. However, CDNV_{sIIN}⁺/IFN-γ⁺ was statistically more efficient at concentrations of 2.7×10¹⁰ and 8.1×10¹⁰ CDNVs. Expectedly, CDNVs derived from mature, SIINFEKL-presenting DC2.4 cells were observed to be more potent activators of CD8⁺ T cells than CDNVs derived from their immature counterparts. Although CDNVs can activate CD8⁺ T cells through a direct mechanism, indirect activation of T cells through uptake and presentation by bystander DC2.4 cells was far more efficient. Results of DC2.4-mediated indirect activation suggest that CDNV_{sIIN}⁺/IFN-γ⁺ may increase the T cell activation efficiency of recipient DC2.4 cells through two ways: (1) inducing maturation of recipient DCs or (2) increased level of SIINFEKL-MHC class I delivered for cross-presentation or cross-dressing of recipient DCs.

To investigate if the increased efficacy of CD8⁺ T cell activation by DC2.4 cells incubated with CDNV_{sIIN}⁺/IFN-γ⁺ is the result of the mature phenotype of precursor cells, DC2.4 cells were treated with CDNV_{Mix}. When treated with DC2.4 cells pretreated with CDNV_{Mix} (FIG. 8B, bottom row) at doses of 1×10⁹, 3×10⁹, and 9×10⁹, CD8⁺ T cell activation remained relatively low and constant, as CD69 MFI_{Geo} was observed to be 1090±29, 1027±45, and 1094±125, respectively, much like treatment with CDNV_{SIIN}⁺/IFN-γ⁻. The number of CD8⁺ T cells with elevated CD69 expression slightly rose following treatment with DC2.4 cells pretreated with 2.7×10¹⁰ (CD69 MFI_{Geo} of 1200±100) CDNV_{Mix}, while CD8⁺ T cell activation significantly increased upon treatment with DC2.4 cells pretreated with 8.1×10¹⁰ (MFI_{Geo} of 4300±300) CDNV_{Mix}.

CDNV_{SIIN}⁺/IFN-γ⁻ was statistically more efficient at indirect T cell activation than CDNV_{Mix}, but only at the 8.1×10¹⁰ CDNV dose. Despite the presence of CDNV_{SIIN}/IFN-γ⁺ to assist in any potential activation of recipient DC2.4 cells, and similar to results observed in the direct activation assay, CD8⁺ T cell activation by CDNV_{Mix} more closely resembled treatment with CDNV_{SIIN}⁺/IFN-γ⁻ than CDNV_{SIIN}/IFN-γ⁺. Activation of CD8⁺ T cells by bystander DC2.4 cells was less efficient when DC2.4 cells were treated with CDNV_{Mix}, suggesting that CDNV_{IFN}-γ⁺ may not activate recipient DC2.4 cells, but instead, mature CDNVs may deliver increased SIINFEKL in complex with MHC class I molecules for cross-presentation or cross-dressing that results in more efficient activation of CD8⁺ T cells than CDNV_{SIIN}/IFN-γ

### CDNVs and Bystander DCs

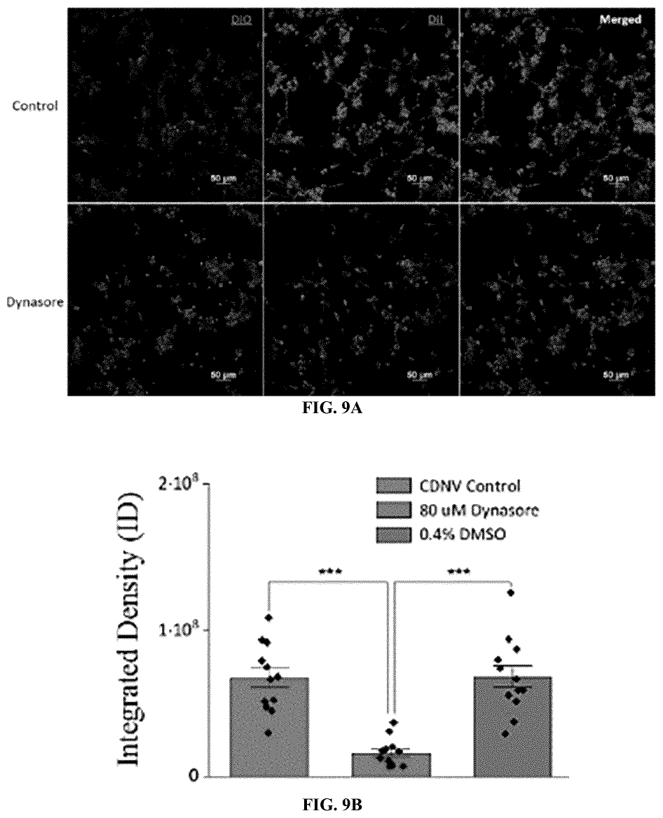

To investigate the mechanism of DC-CDNV interaction with bystander DC2.4 cells, DC2.4 cells were labeled with lipophilic fluorescent label DiO and incubated with 5×10¹⁰ CDNVs, labeled with DiI, for 3 h with or without the clathrin-mediated endocytosis inhibitor Dynasore, which blocks≥90% of endocytosis at a concentration of 80 μM. (58) Cells in 0.4% DMSO were used as a control. Cells were imaged by confocal microscopy, and DiI-labeled CDNV uptake was calculated using integrated density (ID), i.e., the product of area in pixels and mean gray value. As shown in FIG. 9A-9B, untreated DC2.4 cells had an ID of 6.7×10⁷±8×10⁶, while the uptake of DiI-labeled CDNVs by DC2.4 in the presence of 80 μM Dynasore significantly decreased, as ID for cells treated with Dynasore was observed to be 1.6×10⁷±3×10⁶. The presence of DMSO was not observed to influence CDNV uptake by DC2.4 cells, as DiI-labeled CNDV uptake by untreated DC2.4 cells was similar to that by DC2.4 cells treated with 0.4% DSMO (ID of 6.8×10⁸±9×10⁶). As CDNV uptake by untreated cells was significantly higher than that by cells treated with Dynasore, the uptake of CDNVs by recipient bystander DC2.4 cells primarily occurs through a clathrin-mediated route.

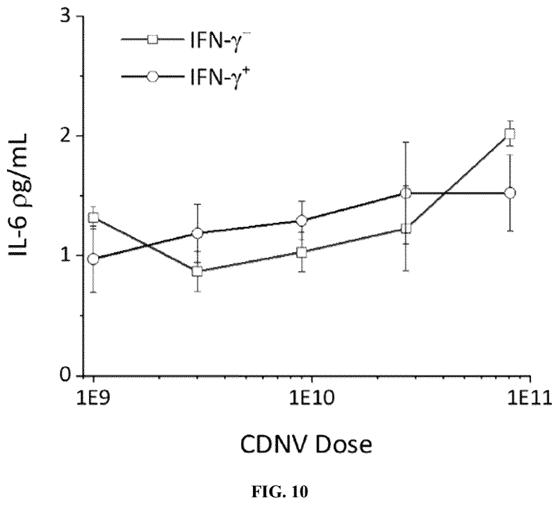

It has previously been reported that CDNVs generated from macrophages are capable of mediating repolarization between pro- and anti-inflammatory states of recipient macrophage cells. (42) With this in mind, the effect DC2.4 CDNVs have on the activation of recipient DC2.4 cells was examined (FIG. 10). To investigate if DC2.4-derived CDNVs are capable of inducing maturation of recipient bystander APCs, DC2.4 cells were treated with CDNVs generated from immature (CDNV_{IFN}-γ⁻) or IFN-γ-induced mature (CDNV_{IFN}-γ) parental cells for 24 h and interleukin-6 (IL-6) production was measured by ELISA. Although a trend of increased IL-6 production with increasing CDNV dose was observed with DC2.4 cells treated with both CDNV_{IFN}-γ⁻ and CDNV_{IFN}-γ⁺, IL-6 production did not significantly differ across the dose range or between CDNV_{IFN}-γ⁻- and CDNV_{IFN}-γ⁺-treated DC2.4. These results suggest that CDNVs originating from IFN-γ-induced mature DC2.4 cells lack the ability to induce maturation of immature recipient DC2.4 cells.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Chaplin, D. D. Overview of the immune response. J. Allergy Clin. Immunol. 2010, 125, S3-S23, DOI: 10.1016/j.jaci.2009.12.980
2. Gil-Torregrosa, B. C.; Lennon-Duménil, A. M.; Kessler, B.; Guermonprez, P.; Ploegh, H. L.; Fruci, D.; van Endert, P.; Amigorena, S. Control of cross-presentation during dendritic cell maturation. Eur. J. Immunol. 2004, 34, 398-407, DOI: 10.1002/eji.200324508
3. Brossart, P.; Wirths, S.; Stuhler, G.; Reichardt, V. L.; Kanz, L.; Brugger, W. Induction of cytotoxic T-lymphocyte responses in vivo after vaccinations with peptide-pulsed dendritic cells. Blood 2000, 96, 3102-3108, DOI: 10.1182/blood. V96.9.3102
4. Shenoda, B. B.; Ajit, S. K. Modulation of Immune Responses by Exosomes Derived from Antigen-Presenting Cells. Clin. Med. Insights: Pathol. 2016, 9s1,1-8, DOI: 10.4137/CPath.S39925
5. Pitt, J. M.; Charrier, M.; Viaud, S.; André, F.; Besse, B.; Chaput, N.; Zitvogel, L. Dendritic Cell-Derived Exosomes as Immunotherapies in the Fight against Cancer. J. Immunol. 2014, 193, 1006-1011, DOI: 10.4049/jimmunol.1400703
6. Beit-Yannai, E.; Tabak, S.; Stamer, W. D. Physical exosome:exosome interactions. J. Cell. Mol. Med. 2018, 22, 2001-2006, DOI: 10.1111/jcmm. 13479
7. Hartjes, T. A.; Mytnyk, S.; Jenster, G. W.; van Steijn, V.; van Royen, M. E. Extracellular Vesicle Quantification and Characterization: Common Methods and Emerging Approaches. Bioengineering (Basel) 2019, 6,7, DOI: 10.3390/bioengineering6010007
8. Tkach, M.; Théry, C. Communication by Extracellular Vesicles: Where We Are and Where We Need to Go. Cell 2016, 164, 1226-1232, DOI: 10.1016/j.cell.2016.01.043
9. Mathivanan, S.; Ji, H.; Simpson, R. J. Exosomes: extracellular organelles important in intercellular communication. J. Proteomics 2010, 73, 1907-1920, DOI: 10.1016/j.jprot.2010.06.006
10. Mulcahy, L. A.; Pink, R. C.; Carter, D. R. F. Routes and mechanisms of extracellular vesicle uptake. J. Extracell. Vesicles 2014, 3, 24641, DOI: 10.3402/jev.v3.24641
11. Xia, X.; Wang, Y.; Huang, Y.; Zhang, H.; Lu, H.; Zheng, J. C. Exosomal miRNAs in central nervous system diseases: biomarkers, pathological mediators, protective factors and therapeutic agents. Prog. Neurobiol. 2019, 183, 101694 DOI: 10.1016/j.pneurobio.2019.101694
12. El Andaloussi, S.; Lakhal, S.; Mäger, I.; Wood, M. J. A. Exosomes for targeted siRNA delivery across biological barriers. Adv. Drug Delivery Rev. 2013, 65, 391-397, DOI: 10.1016/j.addr.2012.08.008
13. Man, K.; Brunet, M. Y.; Jones, M.-C.; Cox, S. C. Engineered Extracellular Vesicles: Tailored-Made Nanomaterials for Medical Applications. Nanomaterials (Basel) 2020, 10, 1838, DOI: 10.3390/nano10091838
14. Sun, D.; Zhuang, X.; Zhang, S.; Deng, Z.-B.; Grizzle, W.; Miller, D.; Zhang, H.-G. Exosomes are endogenous nanoparticles that can deliver biological information between cells. Adv. Drug Delivery Rev. 2013, 65, 342-347, DOI: 10.1016/j.addr.2012.07.002
15. Morelli, A. E.; Larregina, A. T.; Shufesky, W. J.; Sullivan, M. L.; Stolz, D. B.; Papworth, G. D.; Zahorchak, A. F.; Logar, A. J.; Wang, Z.; Watkins, S. C.; Falo, L. D., Jr.; Thomson, A. W. Endocytosis, intracellular sorting, and processing of exosomes by dendritic cells. Blood 2004, 104, 3257-3266, DOI: 10.1182/blood-2004-03-0824
16. Das, C. K.; Jena, B. C.; Banerjee, I.; Das, S.; Parekh, A.; Bhutia, S. K.; Mandal, M. Exosome as a Novel Shuttle for Delivery of Therapeutics across Biological Barriers. Mol. Pharmaceutics 2019, 16, 24-40, DOI: 10.1021/acs.molpharmaceut.8b00901
17. Huppa, J. B.; Davis, M. M. T-cell-antigen recognition and the immunological synapse. Nat. Rev. Immunol. 2003, 3, 973-983, DOI: 10.1038/nri1245
18. Théry, C.; Ostrowski, M.; Segura, E. Membrane vesicles as conveyors of immune responses. Nat. Rev. Immunol. 2009, 9, 581-593, DOI: 10.1038/nri2567
19. Greening, D. W.; Gopal, S. K.; Xu, R.; Simpson, R. J.; Chen, W. Exosomes and their roles in immune regulation and cancer. Semin. Cell Dev. Biol. 2015, 40, 72-81, DOI: 10.1016/j.semcdb.2015.02.009
20. Pitt, J. M.; Kroemer, G.; Zitvogel, L. Extracellular vesicles: masters of intercellular communication and potential clinical interventions. J. Clin. Invest. 2016, 126, 1139-1143, DOI: 10.1172/JCI87316
21. Chaput, N.; Théry, C. Exosomes: immune properties and potential clinical implementations. Semin. Immunopathol. 2011, 33, 419-440, DOI: 10.1007/s00281-010-0233-9
22. Zitvogel, L.; Regnault, A.; Lozier, A.; Wolfers, J.; Flament, C.; Tenza, D.; Ricciardi-Castagnoli, P.; Raposo, G.; Amigorena, S. Eradication of established murine tumors using a novel cell-free vaccine: dendritic cell derived exosomes. Nat. Med. 1998, 4, 594-600, DOI: 10.1038/nm0598-594
23. Robbins, P. D.; Morelli, A. E. Regulation of immune responses by extracellular vesicles. Nat. Rev. Immunol. 2014, 14, 195-208, DOI: 10.1038/nri3622
24. Dooley, K.; McConnell, R.; Xu, K.; Lewis, N.; Haupt, S.; Youniss, M.; Martin, S.; Sia, C.; McCoy, C.; Moniz, R.; Burenkova, O.; Sanchez-Salazar, J.; Jang, S. C.; Choi, B.; Harrison, R. A.; Houde, D.; Burzyn, D.; Leng, C.; Kirwin, K.; Ross, N. L.; Finn, J. D.; Gaidukov, L.; Economides, K. D.; Estes, S.; Thornton, J. E.; Kulman, J. D.; Sathyanarayanan, S.; Williams, D. E. A Versatile Platform for Generating Engineered Extracellular Vesicles with Defined Therapeutic Properties. Mol. Ther. 2021, 29, 1729, DOI: 10.1016/j.ymthe.2021.01.020
25. Jafari, D.; Shajari, S.; Jafari, R.; Mardi, N.; Gomari, H.; Ganji, F.; Forouzandeh Moghadam, M.; Samadikuchaksaraei, A. Designer Exosomes: A New Platform for Biotechnology Therapeutics. BioDrugs 2020, 34, 567-586, DOI: 10.1007/s40259-020-00434-x
26 Escudier, B.; Dorval, T.; Chaput, N.; André, F.; Caby, M. P.; Novault, S.; Flament, C.; Leboulaire, C.; Borg, C.; Amigorena, S. Vaccination of metastatic melanoma patients with autologous dendritic cell (DC) derived-exosomes: results of thefirst phase I clinical trial. J. Transl. Med. 2005, 3, 10, DOI: 10.1186/1479-5876-3-10
27 Morse, M. A.; Garst, J.; Osada, T.; Khan, S.; Hobeika, A.; Clay, T. M.; Valente, N.; Shreeniwas, R.; Sutton, M. A.; Delcayre, A. A phase I study of dexosome immunotherapy in patients with advanced non-small cell lung cancer. J. Transl. Med. 2005, 3, 9, DOI: 10.1186/1479-5876-3-9
28. Besse, B.; Charrier, M.; Lapierre, V.; Dansin, E.; Lantz, O.; Planchard, D.; Le Chevalier, T.; Livartoski, A.; Barlesi, F.; Laplanche, A.Dendritic cell-derived exosomes as maintenance immunotherapy after first line chemotherapy in NSCLC. OncoImmunology 2016, 5, e1071008 DOI: 10.1080/2162402X.2015.1071008
29 Soltani, F.; Parhiz, H.; Mokhtarzadeh, A.; Ramezani, M. Synthetic and Biological Vesicular Nano-Carriers Designed for Gene Delivery. Curr. Pharm. Des. 2015, 21, 6214-6235, DOI: 10.2174/1381612821666151027153410
30 Vader, P.; Mol, E. A.; Pasterkamp, G.; Schiffelers, R. M. Extracellular vesicles for drug delivery. Adv. Drug Delivery Rev. 2016, 106, 148-156, DOI: 10.1016/j.addr.2016.02.006
31. Min, Y.; Caster, J. M.; Eblan, M. J.; Wang, A. Z. Clinical Translation of Nanomedicine. Chem. Rev. 2015, 115, 11147-11190, DOI: 10.1021/acs.chemrev.5b00116
32 Sukhanova, A.; Bozrova, S.; Sokolov, P.; Berestovoy, M.; Karaulov, A.; Nabiev, I. Dependence of Nanoparticle Toxicity on Their Physical and Chemical Properties. Nanoscale Res. Lett. 2018, 13, 44, DOI: 10.1186/s11671-018-2457-x
33. Szebeni, J.; Moghimi, S. M. Liposome triggering of innate immune responses: a perspective on benefits and adverse reactions. J. Liposome Res. 2009, 19, 85-90, DOI: 10.1080/08982100902792855
34. Xu, C.; Ju, D.; Zhang, X. Cell Membrane-Derived Vesicle: A Novel Vehicle for Cancer Immunotherapy. Front. Immunol. 2022, 13, 923598 DOI: 10.3389/fimmu.2022.923598
35. Zeng, Y.; Li, S.; Zhang, S.; Wang, L.; Yuan, H.; Hu, F. Cell membrane coated-nanoparticles for cancer immunotherapy. Acta Pharm. Sin. B 2022, 12, 3233-3254, DOI: 10.1016/j.apsb.2022.02.023
36 Cheng, S.; Xu, C.; Jin, Y.; Li, Y.; Zhong, C.; Ma, J.; Yang, J.; Zhang, N.; Li, Y.; Wang, C.Artificial Mini Dendritic Cells Boost T Cell-Based Immunotherapy for Ovarian Cancer. Adv. Sci. (Weinh) 2020, 7, 1903301, DOI: 10.1002/advs.201903301
37 Xu, C.-H.; Ye, P.-J.; Zhou, Y.-C.; He, D.-X.; Wei, H.; Yu, C.-Y. Cell membrane-camouflaged nanoparticles as drug carriers for cancer therapy. Acta Biomater. 2020, 105, 1-14, DOI: 10.1016/j.actbio.2020.01.036
38 Ochyl, L. J.; Moon, J. J. Dendritic Cell Membrane Vesicles for Activation and Maintenance of Antigen-Specific T Cells. Adv. Healthcare Mater. 2019, 8, e1801091 DOI: 10.1002/adhm.201801091
39 Kovar, M.; Boyman, O.; Shen, X.; Hwang, I.; Kohler, R.; Sprent, J. Direct stimulation of T cells by membrane vesicles from antigen-presenting cells. Proc. Natl. Acad. Sci. U.S.A 2006, 103, 11671-11676, DOI: 10.1073/pnas.0603466103
40. Sprent, J. Direct stimulation of naïve T cells by antigen-presenting cell vesicles. Blood Cells Mol., Dis. 2005, 35, 17-20, DOI: 10.1016/j.bcmd.2005.04.004
41. Théry, C.; Duban, L.; Segura, E.; Véron, P.; Lantz, O.; Amigorena, S. Indirect activation of naïve CD4⁺ T cells by dendritic cell-derived exosomes. Nat. Immunol. 2002, 3, 1156-1162, DOI: 10.1038/ni854
42. Neupane, K. R.; McCorkle, J. R.; Kopper, T. J.; Lakes, J. E.; Aryal, S. P.; Abdullah, M.; Snell, A. A.; Gensel, J. C.; Kolesar, J.; Richards, C. I. Macrophage-Engineered Vesicles for Therapeutic Delivery and Bidirectional Reprogramming of Immune Cell Polarization. ACS Omega 2021, 6, 3847-3857, DOI: 10.1021/acsomega.0c05632
43. Zhou, M.; Philips, M. R. Nitrogen Cavitation and Differential Centrifugation Allows for Monitoring the Distribution of Peripheral Membrane Proteins in Cultured Cells. JoVE 2017, 126, e56037 DOI: 10.3791/56037
44. Burden, D. W. Guide to the disruption of biological samples—2012. Random Primers 2012, 12, 1-25
45 Snell, A. A.; Neupane, K. R.; McCorkle, J. R.; Fu, X.; Moonschi, F. H.; Caudill, E. B.; Kolesar, J.; Richards, C. I. Cell-Derived Vesicles for in Vitro and in Vivo Targeted Therapeutic Delivery. ACS Omega 2019, 4, 12657-12664, DOI: 10.1021/acsomega.9b01353
46. Benvenuti, F.; Lagaudrière-Gesbert, C.; Grandjean, I.; Jancic, C.; Hivroz, C.; Trautmann, A.; Lantz, O.; Amigorena, S. Dendritic cell maturation controls adhesion, synapse formation, and the duration of the interactions with naïve T lymphocytes. J. Immunol. 2004, 172, 292-301, DOI: 10.4049/jimmunol.172.1.292
47. Mbongue, J. C.; Nieves, H. A.; Torrez, T. W.; Langridge, W. H. R. The Role of Dendritic Cell Maturation in the Induction of Insulin-Dependent Diabetes Mellitus. Front. Immunol. 2017, 8, 327, DOI: 10.3389/fimmu.2017.00327
48. Alloatti, A.; Kotsias, F.; Magalhaes, J. G.; Amigorena, S. Dendritic cell maturation and cross-presentation: timing matters !. Immunol. Rev. 2016, 272, 97-108, DOI: 10.1111/imr.12432
49. Verdijk, P.; van Veelen, P. A.; de Ru, A. H.; Hensbergen, P. J.; Mizuno, K.; Koerten, H. K.; Koning, F.; Tensen, C. P.; Mommaas, A. M. Morphological changes during dendritic cell maturation correlate with cofilin activation and translocation to the cell membrane. Eur. J. Immunol. 2004, 34, 156-164, DOI: 10.1002/eji.200324241
50. Kim, M. K.; Kim, J. Properties of immature and mature dendritic cells: phenotype, morphology, phagocytosis, and migration. RSC Adv. 2019, 9, 11230-11238, DOI: 10.1039/c9ra00818 g
51. Segura, E.; Nicco, C.; Lombard, B.; Véron, P.; Raposo, G.; Batteux, F.; Amigorena, S.; Théry, C. ICAM-1 on exosomes from mature dendritic cells is critical for efficient naïve T-cell priming. Blood 2005, 106, 216-223, DOI: 10.1182/blood-2005-01-0220
52. Yamakita, Y.; Matsumura, F.; Lipscomb, M. W.; Chou, P. C.; Werlen, G.; Burkhardt, J. K.; Yamashiro, S. Fascin1 promotes cell migration of mature dendritic cells. J. Immunol. 2011, 186, 2850-2859, DOI: 10.4049/jimmunol.1001667
53. Chernyshev, V. S.; Rachamadugu, R.; Tseng, Y. H.; Belnap, D. M.; Jia, Y.; Branch, K. J.; Butterfield, A. E.; Pease, L. F., III; Bernard, P. S.; Skliar, M. Size and shape characterization of hydrated and desiccated exosomes. Anal. Bioanal. Chem. 2015, 407, 3285-3301, DOI: 10.1007/s00216-015-8535-3
54. Chuo, S. T.-Y.; Chien, J. C.-Y.; Lai, C. P.-K. Imaging extracellular vesicles: current and emerging methods. J. Biomed. Sci. Eng. 2018, 25, 91, DOI: 10.1186/s12929-018-0494-5
55. Wu, Y.; Deng, W.; Klinke, D. J., II Exosomes: improved methods to characterize their morphology, RNA content, and surface protein biomarkers. Analyst 2015, 140, 6631-6642, DOI: 10.1039/c5an00688k
56. González-Amaro, R.; Cortés, J. R.; Sánchez-Madrid, F.; Martín, P. Is CD69 an effective brake to control inflammatory diseases ?. Trends Mol. Med. 2013, 19, 625, DOI: 10.1016/j.molmed.2013.07.006
57 Hood, J. L. The association of exosomes with lymph nodes. Semin. Cell Dev. Biol. 2017, 67, 29-38, DOI: 10.1016/j.semcdb.2016.12.002
58. Kirchhausen, T.; Macia, E.; Pelish, H. E. Use of Dynasore, the Small Molecule Inhibitor of Dynamin, in the Regulation of Endocytosis. In Methods in Enzymology; Vol. 438; Academic Press, 2008; pp. 77-93.
59 Laureano R S, Sprooten J, Vanmeerbeerk I, Borras D M, Govaerts J, Naulaerts S, Berneman Z N, Beuselinck B, Bol K F, Borst J, Coosemans A, Datsi A, Fučíková J, Kinget L, Neyns B, Schreibelt G, Smits E, Sorg R V, Spisek R, Thielemans K, Tuyaerts S, De Vleeschouwer S, de Vries I J M, Xiao Y, Garg A D. Trial watch: Dendritic cell (DC)-based immunotherapy for cancer. Oncoimmunology. 2022 Jul. 4; 11(1):2096363. doi: 10.1080/2162402X.2022.2096363. PMID: 35800158; PMCID: PMC9255073.
60. Chometon, T. Q., Siqueira, M. d. S., Sant′anna, J. C., Almeida, M. R., Gandini, M., Martins de Almeida Nogueira, A. C., Araújo-Jorge, T. C. (2020). A protocol for rapid monocyte isolation and generation of singular human monocyte-derived dendritic cells. PLOS ONE, 15(4), e0231132. doi.org/10.1371/journal.pone.0231132.
61. Lutz, M. B., Schuler, G., & Steinkasserer, A. Guidelines for mouse and human DC generation. Eur J Immunol. 2023 Nov.;53(11):e2249816. doi: 10.1002/eji.202249816. Epub 2022 Oct. 27. PMID: 36303448; PMCID: PMC10704271. DOI: 10.1002/eji.202249816
62. Kranz, L., Diken, M., Haas, H. et al. Systemic RNA delivery to dendritic cells exploits antiviral defence for cancer immunotherapy. Nature 534, 396-401 (2016). doi.org/10.1038/nature18300
63. U.S. Pat. No. 10,973,765 to Gho, et al. for “Cell membrane-derived nanovesicles and use thereof.”
64U.S. Pat. No. 9,107,878 to Holaday for “Methods of treating cancer.”
65. Japanese Patent No. JP6235085 for “Means and methods for activated cell cancer immunotherapy using tumor cells and dendritic cells killed by high hydrostatic pressure.”
66. U.S. Patent Application Publication No. 2021/0220458 by Kalantarov, et al. for “Development of dual whole cell-based vaccine against pancreatic cancer.”

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of making an antigen-presenting dendritic cell membrane derived nanovesicle (CDNV), comprising:
(a) incubating a dendritic cell (DC) with an antigen and an agent to activate the DC, thereby generating a mature DC displaying a major histocompatibility complex class I (MHC) presenting the antigen; and
(b) fragmenting the membrane of the mature DC and allowing the fragmented membrane to assemble into a CDNV displaying the MHC presenting the antigen.

2. The method of claim 1, wherein the antigen is presented by a target cell.

3. The method of claim 2, wherein the target cell is a tumor cell.

4. The method of claim 3, wherein the target cell has been obtained from an in vivo environment at a site of a tumor in a subject.

5. The method of claim 2, wherein the target cell is a cell infected with a pathogen.

6. The method of claim 3, wherein the target cell has been obtained from an in vivo environment at a site of an infection in a subject.

7. The method of claim 2, and further comprising obtaining the target cell from an in vitro environment or an in vivo environment.

8. The method of claim 1, and further comprising obtaining the DC from a circulating hematopeotic cell obtained from a subject.

9. The method of claim 1, wherein fragmenting comprises exposing the mature DC to sonication.

10. The method of claim 1, wherein fragmenting comprises pressurizing the mature DC under nitrogen and releasing pressure.

11. The method of claim 1, and further comprising suspending the membrane fragments of the mature DC in an assembly solution comprising cargo such that the CDNV encapsulates the cargo during assembly.

12. The method of claim 11, wherein the cargo is selected from the group consisting of genetic material, therapeutic agent, protein, and fluorescent marker.

13. A method of activating an antigen-specific T cell, comprising:
(a) incubating a dendritic cell (DC) with an antigen and an agent to activate the DC, thereby generating a mature DC displaying a major histocompatibility complex class I (MHC) presenting the antigen;
(b) fragmenting the membrane of the mature DC and allowing the fragmented membrane to assemble into a CDNV displaying the MHC presenting the antigen; and
(c) delivering the CDNV to an environment including the T cell, thereby directly activating the T cell, or indirectly activating the T cell through a bystander antigen presenting cell (APC) that uptakes the CDNV and presents the antigen.

14. The method of claim 13, wherein the antigen is presented by a target cell.

15. The method of claim 14, wherein the target cell is a tumor cell or a cell infected with a pathogen.

16. The method of claim 15, wherein the target cell has been obtained from an in vivo environment at a site of a tumor or an infection in a subject.

17. The method of claim 13, wherein fragmenting comprises exposing the mature DC to sonication.

18. The method of claim 13, wherein fragmenting comprises pressurizing the mature DC under nitrogen and releasing pressure.

19. The method of claim 13, and further comprising suspending the membrane fragments of the mature DC in an assembly solution comprising cargo such that the CDNV encapsulates the cargo during assembly.

20. The method of claim 13, and further comprising administering the CDNV to the subject.
